(19) United States
(12) Patent Application Publication
(10) Pub. No.: US 2006/0099273 A1
(43) Pub. Date: May 11, 2006

(54) Sterile preparations and compositions including stinging capsules and methods of producing and using same

(75) Inventor: Tamar Lotan, Jordan Valley (IL)

Correspondence Address: Martin Moynihan;c/o ANTHONY CASTORINA, SUITE 207, 2001 JEFFERSON DAVIS HIGHWAY, ARLINGTON, VA, 22202, US

(73) Assignee: NanoCyte Inc.

(21) Appl. No.: 10/981,522

(22) Filed: Nov. 5, 2004

(51) Int. Cl. A61K 35/12

(52) U.S. Cl. 424/529

(57) ABSTRACT

A sterile stinging capsule preparation and methods of producing and using same are provided.

### FIELD AND BACKGROUND OF THE INVENTION

The present invention relates to production of sterile stinging capsule preparations and their use in delivering a therapeutic, diagnostic or cosmetic agent into a tissue.

Therapeutic agents such as drugs are a mainstay of modern medicine and are used for the prevention, diagnosis, alleviation, treatment, or cure of diseases.

Biological, biochemical and/or physical barriers often limit delivery of therapeutic agents to target tissue. For example, skin and/or various organ membranes are physical barriers, which must be traversed by a topically administered drug targeted at internal tissues. Orally administered drugs must be resistant to the low pH conditions and digestive enzymes present in the gastrointestinal (GI) tract.

To traverse such barriers, drugs targeted at internal tissues are often administered via a transdermal injection, using a syringe and a needle or other mechanical devices. A transdermal injection delivers drugs into the subcutaneous space thus traversing the epidermis-dermis layers.

Anatomically, the skin of a human body is subdivided into three compartments: an epidermis, a dermis and a subcutaneous layer, of which the epidermis plays a key role in blocking drug delivery via the skin (the dourest layer of the epidermis is the stratum corneum which is called also the horny layer). The epidermis is 0.1 mm or more in thickness and consists mainly of protein surrounded by lipid, thus rendering the epidermis hydrophobic.

Although the syringe and needle is an effective delivery device, it is sensitive to contamination, while use thereof is often accompanied by pain and/or bruising. In addition, the use of such a device is accompanied by risk of accidental needle injury to a health care provider.

Mechanical injection devices based on compressed gasses have been developed to overcome the above-mentioned limitations of syringe and needle devices. Such devices typically utilize compressed gas (such as, helium or carbon dioxide) to deliver medications at high velocity through a narrow aperture.

Although such devices traverses some of the limitations mentioned above, their efficiency is medication dependent, and their use can lead to pain, bruising and lacerations.

Other less common delivery methods utilize a pulsed Yag laser to punctuate the stratum corneum in order to deliver medication via diffusion and enhancement of ionic compound flux across the skin by the application of an electric current. Although such methods are effective in delivering small charged molecules, a danger of skin burns accompanies their use.

Non-invasive methods, which overcome some of the limitations inherent to the invasive delivery methods described above, have also been described. Such methods utilize preparations, which include an active ingredient disposed within lipid vehicles (e.g., liposomes) or micelles or accompanied with skin permeation agent such that absorption of the active ingredient through the skin is enhanced. Such preparations can be directly applied to a skin region or delivered via transdermal devices such as membranes, pressure-sensitive adhesive matrices and skin patches.

In transdermal delivery, the active ingredient penetrates the skin and enters the capillary blood or the lymph circulation system, which carries the drug to the target organ or to the tissue or has a local effect.

For several years, transdermal drug delivery systems have been employed to effectively introduce a limited number of drugs through unbroken skin. Aside from comfort and convenience, transdermal systems avoid the barriers, delivery rate control problems and potential toxicity concerns associated with traditional administration techniques, such as oral, intramuscular or intravenous delivery.

Although transdermal delivery offers an alternative to some invasive delivery methods, the efficiency thereof is affected by the physical and chemical properties of a drug and physiological or pathological parameters such as the skin hydration, temperature, location, injury, and the body metabolism.

Many limitations of invasive and non-invasive delivery devices may be circumvented by the use of “stinging capsules” (e.g., cnidocysts, nematocysts and polar capsules) isolated therefrom for tissue delivery of a therapeutic, diagnostic or cosmetic agents.

Cnidaria (hydras, sea anemones, jellyfish and corals) are aquatic animals, which possess a variety of compounds which are stored and delivered via specialized capsules (cnidocysts), which form a part of specialized cells termed stinging cells (cnidocytes, nematocytes, ptychocytes and the like). The stinging capsules act as microscopic syringes and serve as a prey or defense mechanism. The Cnidaria family which encompasses 10,000 known species, includes sedentary single or colonial polyps and pelagic jellyfish. In some of these species, cnidocytes account for more than 45% of the cells present (Tardent 1995).

Discharge is initiated by a rapid osmotic influx of water which generates an internal hydrostatic (liquid) pressure of 150 atmospheres forcing capsule rupture and ejection of the tubule (Holstein and Tardent 1984). During ejection, the long coiled and twisted tubule is averted and its length increases by 95 percent. Accelerating at 40,000 g, the tubule untwists to generate a torque force, which rotates the tubule several times around its axis. These mechanical processes generate a powerful driving force, which enables efficient delivery of the compounds, the toxins and enzymes stored within the capsule (Lotan et al. 1995, 1996; Tardent 1995). This process, which occurs within microseconds, is among the most rapid exocytosis events in biology (Holstein and Tardent 1984).

There are at least three dozen known types of cnidocysts (also termed cnidae) including more than 30 varieties of nematocysts found in most Cnidaria and spirocysts, and ptychocysts found mainly in the Cnidaria class Anthozoa (Mariscal 1974).

Accordingly, U.S. Pat. No. 6,163,344 and U.S. patent application Ser. Nos. 10/406,202 and 09/963,672 to the present inventors teach the use of stinging capsules or cells for the purpose of rapidly and efficiently delivering therapeutic, cosmetic or diagnostic agents into a target tissue.

Since stinging capsules utilized for delivering agents into a tissue may harbor potentially infectious microorganisms (either naturally occurring or contaminants), the present inventors sought approaches which can be utilized to effectively inactivate microbial contaminants harboring stinging capsules, rendering such capsules more suitable for therapeutic applications. While reducing the present invention to practice, the present inventors have surprisingly and unexpectedly uncovered that stinging capsules subjected to harsh physical and chemical conditions retain their discharging activity, thereby generating for the first time aseptic stinging capsules which are highly suitable for use in delivering therapeutic, diagnostic or cosmetic agents to a tissue.

### SUMMARY OF THE INVENTION

According to one aspect of the present invention there is provided a composition-of-matter comprising a sterile stinging capsule preparation including at least one stinging capsule.

According to another aspect of the present invention there is provided a pharmaceutical composition including, as an active ingredient, a stinging capsule preparation including at least one stinging capsule and a pharmaceutically acceptable carrier, the pharmaceutical composition being devoid of viable microorganisms.

According to yet another aspect of the present invention there is provided a method of preparing a sterile stinging capsule preparation. The method includes providing a preparation including at least one stinging capsule and subjecting the preparation to sterilizing conditions.

According to still another aspect of the present invention there is provided a method of delivering a therapeutic, diagnostic or cosmetic agent into a tissue. The method includes the steps of (i) applying to the tissue a pharmaceutical composition, which includes a sterile stinging capsule preparation which includes at least one stinging capsule and the therapeutic, diagnostic or cosmetic agent, and (ii) triggering a discharge of the at least one stinging capsule.

According to an additional aspect of the present invention there is provided a method of delivering a therapeutic, diagnostic or cosmetic agent into a tissue. The method includes the steps of (i) applying onto an outer surface of the tissue a therapeutic, diagnostic or cosmetic agent, (ii) applying onto the outer surface of the tissue a sterile stinging capsule preparation including at least one stinging capsule, and (iii) triggering a discharge of the at least one stinging capsule.

According to yet an additional aspect of the present invention there is provided a stinging capsule preparation having an assurance of acceptable bioburden lower than 1 cfu/ml as determined by the standard method of ISO 11737 part 1 BFEN 2274-3.

According to further features in preferred embodiments of the invention described below, the sterile stinging capsule preparation is devoid of a microorganism capable of growing in a microbiological culture medium.

According to still further features in the described preferred embodiments the microbiological culture medium is soybean casein digest broth (SCDB) or fluid thioglycollate medium (FTM).

According to still further features in the described preferred embodiments, the composition-of-matter further including a therapeutic, diagnostic or cosmetic agent.

According to still further features in the described preferred embodiments the therapeutic, diagnostic or cosmetic agent is disposed in a liquid surrounding, or stored within, the at least one stinging capsule.

According to still further features in the described preferred embodiments the cosmetic agent is selected from the group consisting of a cosmetic dye, an anti wrinkling agent, an anti-acne agent, a vitamin, a skin peel agent, a hair follicle stimulating agent and a hair follicle suppressing agent.

According to still further features in the described preferred embodiments the therapeutic agent is selected from the group consisting of a drug, a nucleic acid construct, a vaccine, a hormone, an enzyme and an antibody.

According to still further features in the described preferred embodiments the therapeutic agent is a prodrug activatable prior to, during or following discharge of the at least one sterile stinging capsule.

According to still further features in the described preferred embodiments the at least one stinging capsule is capable of delivering the therapeutic, diagnostic or cosmetic agent into a tissue.

According to still further features in the described preferred embodiments an endogenous toxin naturally stored within the at least one stinging capsule is substantially non-toxic to mammals.

According to still further features in the described preferred embodiments an endogenous toxin naturally stored within the at least one stinging capsule is substantially non-toxic to mammals.

According to still further features in the described preferred embodiments the endogenous toxin is non-functional.

According to still further features in the described preferred embodiments the at least one stinging capsule is derived from an organism of a class selected from the group consisting of Anthozoa, Hydrozoa and Scyphozoa.

According to still further features in the described preferred embodiments the at least one stinging capsule is derived from an organism of a phylum selected from the group consisting of Cnidaria, Dinoflagellata and Myxozoa.

According to still further features in the described preferred embodiments the composition-of-matter or pharmaceutical composition further comprises an effective amount of at least one preservative capable of preventing microbial growth within the sterile stinging capsule preparation.

According to still further features in the described preferred embodiments the at least one preservative is selected from the group consisting of ethanol, propylene glycol, hydroxylpropyl cellulose, cetiol, carbopol, PVP and an inorganic salt.

According to still further features in the described preferred embodiments the stinging capsule preparation further comprises a therapeutic, diagnostic or cosmetic agent.

According to still further features in the described preferred embodiments the pharmaceutically acceptable carrier is selected from the group consisting of an aqueous solution, a gel, an oil and semi solid formulation.

According to still further features in the described preferred embodiments the sterilization conditions are effected by a physical sterilization method.

According to still further features in the described preferred embodiments the physical sterilization method is selected from the group consisting of an exposure to moist heat, an exposure to dry heat, an exposure to gamma radiation, an exposure to ultraviolet radiation and an exposure to microwave radiation.

According to still further features in the described preferred embodiments the inactivating is effected by a chemical sterilization method.

According to still further features in the described preferred embodiments the chemical sterilization method is selected from the group consisting of an exposure to ethylene oxide, an exposure to ethanol and an exposure to hydrogen peroxide.

According to still further features in the described preferred embodiments the triggering is effected by a change in pH, a chemical substance, a mechanical force or contact between the at least one sterile stinging capsule and the outer surface of the tissue.

The present invention successfully addresses the shortcomings of the presently known configurations by providing sterile stinging capsules for safe delivery therapeutic, diagnostic or cosmetic agents into a tissue.

### DESCRIPTION OF THE PREFERRED EMBODIMENTS

The present invention is of compositions including stinging capsules (cnidocysts) being devoid of viable microorganisms and methods of producing and utilizing same for delivery of an agent such as, for example, a biologically active agent. Specifically, the present invention relates to the use of sterile stinging capsule preparations for topical, transdermal/intradermal, transmucosal, transcuticular or transmembranal delivery of a therapeutic, diagnostic or cosmetic agent.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

As described in the background section above, U.S. Pat. No. 6,163,344 and U.S. patent application Ser. Nos. 10/406,202 and 09/963,672 teach use of stinging capsules (cnidocysts) or cells for delivering therapeutic, diagnostic or cosmetic agents into a tissue.

The cnidocyst is filled with liquid containing a highly folded inverted tubule. In nature, the cnidocyst discharges and releases its tubule into tissue following physical or chemical triggering. Discharge is initiated by a rapid osmotic influx of water which generates an internal hydrostatic (liquid) pressure of 150 atmospheres forcing capsule rupture and ejection of the tubule (Holstein and Tardent 1984). During ejection, the long coiled and twisted tubule is averted and its length increases several folds. Accelerating at 40,000 g, the tubule untwists to generate a torque force, which rotates the tubule several times around its axis. These mechanical processes generate a powerful driving force, which enables efficient delivery of the natural compounds, (toxins and enzymes) stored within the capsule (Lotan et al. 1995, 1996; Tardent 1995). This process, which occurs within microseconds, is among the most rapid exocytosis events in biology (Holstein and Tardent 1984).

U.S. Pat. No. 6,163,344 and U.S. patent application Ser. Nos. 10/406,202 and 09/963,672 describe compositions which utilize stinging capsules or cells for delivering pharmaceutical or cosmetic agents into a tissue. Since effective procedures for isolating and processing of cnidocysts under aseptic conditions are not known in the prior art and since use of stinging capsules results in penetration of host tissues, the practical use of stinging capsules for delivering pharmaceutical, diagnostic or cosmetic agents intra- or transdermally is hindered by a risk of microbial infection.

Since, as described above, the cnidocyst is a complex and delicate biological organ it was not expected that an isolated cnidocyst could withstand sterilization treatment and still remain operational (capable of discharging and releasing its tubule). However, while reducing the present invention to practice, the present inventors surprisingly and unexpectedly uncovered that isolated stinging capsules can withstand a variety of standard sterilization procedures such as moist heat (autoclaving), gamma irradiation, UV irradiation, microwave irradiation, exposure to ethanol and exposure to hydrogen peroxide and yet maintain their discharging capacity (see Example 1 hereinbelow). The ability to effectively sterilize isolated stinging capsules enables their safe use in delivering therapeutic, diagnostic or cosmetic agents into tissues without the risk of causing microbial infection.

Thus, according to one aspect of the present invention there is provided a composition-of-matter including a preparation of at least one stinging capsule which is devoid of viable microorganisms, such as fungal cells or spores, bacterial cells or spores or viruses. Examples of microorganisms which can contaminate stinging capsule preparations include, but are not limited to, air- and water-borne bacteria and fungi.

The term “viable” when utilized herein with respect to microorganisms refer to ability of the microorganism to grow and/or proliferate.

The stinging capsule of the present invention can be derived from an organism of the phylum Cnidaria, Myxozoa, or Dinoflagellata, preferably it is derived from an organism of the class Anthozoa, Hydrozoa or Scyphozoa. More specifically, the stinging capsule utilized by the present invention can be derived from, for example, subclasses Hexacorallia or Octocorallia of the class Anthozoa, (mostly sea anemone and corals), subclasses Siponophora or Hydroida of the class Hydrozoa, or from subclasses Rhisostomeae or Semastomeae of the class Scyphozoa.

Stinging capsules from such organisms include toxins, which are non-toxic to humans, and other mammals. As such, these stinging capsules isolated therefrom are ideally suited for safe and efficient delivery of a therapeutic, diagnostic or cosmetic agent into mammalian tissue.

It will be appreciated that the use of stinging capsules from organisms which sequester toxins that are not fatal but cause only minor irritations to, for example, mammals, is also envisioned by the present invention.

The stinging capsule of the present invention can be isolated from a cell extract prepared from organs or parts of an organism, which contain the stinging cells (for example a whole hydra or tentacles). Alternatively, stem cells, which give rise to cnidocytes or cnidocysts, can be isolated and cultured or utilized directly.

In addition, stinging capsules from other sources can also be utilized by the present invention provided inactivation of the endogenous toxin is effected prior to use. Such inactivation can be effected via one of several methods such as described in U.S. Pat. No. 6,613,344. Inactivation of endotoxin may also result from a sterilization treatment applied to the stinging capsules, such as moist heat treatment, as further described hereinbelow.

As used herein the term “sterilizafion” refers to use of a physical or chemical procedure which renders microorganisms (e.g., viruses, bacteria, or fungi) non-viable i.e., incapable of growth and/or proliferation. Stinging capsules or cells can be subjected to any one of several physical or chemical treatments in order to generate the sterile stinging capsule preparation of the present invention.

Suitable physical sterilization procedures include, but are not limited to, gamma irradiation (preferably at a dose of at least 0.5 MRad, more preferably 1 MRad, most preferably 2 MRad) ultra violet irradiation (preferably at a wavelength of approximately 312 nm for at least 5 min, more preferably for at least 10 min, most preferably for at least 20 min; 20 min), microwave radiation for at least 2 sec, more preferably for at least 4 sec e.g., most preferably for at least 8 sec), or moist heat (steam under pressure; preferably at a temperature of at least 121° C. for a period of at least 5 min, more preferably at least 10 min, most preferably at least 20 min.

Suitable chemical sterilization procedures include, but not limited to, an exposure to ethyl or isopropyl alcohol (at a concentration of preferably at least 70% w/v, more preferably at least 90%, most preferably at 100%) for a period of at least 1 min, more preferably for at least 5 min, most preferably for at least 10 min; an exposure to hydrogen peroxidase (at a concentration of preferably at least 1%, more preferably at least 2%, most preferably at least 5%) for a period of at least 2 min, more preferably 5, most preferably 15 followed by washing in sterile distilled water or other sterile solution; an exposure to or to ethylene oxide gas (being at a concentration of preferably at least 400 mg/L, more preferably at least 450 mg/L, most preferably at least 500 mg/L at a temperature of at least 55° C.), or an exposure to effective concentrations of glutaraldehyde, formaldehyde, chlorine dioxide, paracetic acid for suitable time periods.

Use of a physical sterilization procedure such as dry or moist heat (e.g., steam) is presently preferred especially in Rhopilema nomadica (jellyfish) since such sterilization conditions will also effectively inactivate the endotoxin harbored within the capsule.

Following sterilization treatment, the preparation is preferably tested for sterility in accordance with regulations and guidelines pertaining to therapeutic compositions. Relevant regulations and guidelines include the US Code of Federal Regulations (CFR) Section 21 and the relevant guidelines of the US Food and Drug Administration (FDA), the US Pharmacopeia (USP), the European Pharmacopeia (EP), the Australian Therapeutic Goods Administration (TGA) and the Japanese Pharmacopeia (JP). Most preferably, the composition of the present invention is tested for sterility according to the specifications of USP Standard 26: 2003 <71>.

The Standard specifies that each manufactured product lot is required to be tested. Each lot is sampled as outlined in Tables 1a-b.

TABLE 1a
Sampling Incidence

| Batch size | Minimum Number to be Tested in Each Media |
|---|---|
| 100 or less | 10% or 4 containers, whichever is greater |
| 101-500 | 10 containers |
| >500 | 2% or 10 containers, whichever is less |
| Bulk - Up to 4 containers | Each container |
| 5-50 | 20% or 4 containers, whichever is greater |
| >50 | 2% or 10, whichever is greater |

TABLE 1b
Sample Volume

| Volume/Container | Minimum Quantity to Test in Each Media |
|---|---|
| Less than 1 mL | The entire contents of each container |
| 1-40 mL | Half the contents of each container but not less |
| | than 1 mL |
| 41-100 mL 20 mL | 20 mL |
| >100 mL | 10% of the contents of the container |

Tested samples are cultured in test media under growth conditions which are conducive for possible contaminating microorganisms. There are two common types of sterility test methods: the direct inoculation (immersion) method and the membrane-filtration method. In the direct inoculation method, the test sample is added to a test growth medium and incubated. Turbidity in the growth medium would indicate microbial contamination. In the membrane-filtration method the sample is filtered through a size exclusion membrane (e.g., 0.22 μm pore size) capable of retaining microorganism, which is then transferred into the growth medium. The membrane-filtration method is advantageous for analyzing large sample volumes or when the sample is colored or turbid and is the sterility test method recommended by the Standard.

In addition, the standard requires to demonstrate the validity of the test method (i.e., that the test sample is devoid of bacteriostatic/fungistatic elements) by recovery of a small number of micro-organisms in the presence of the tested sample. It is preferable to add the challenge organisms directly to the tested sample prior to membrane filtration or direct inoculation. This test is typically required only once for a given sample type, provided that no additional changes to the source, product, formulation or manufacturing process occurred.

The Standard specifies using two growth media: the Soybean Casein Digest Broth (SCDB) and the Fluid Thioglycollate Medium (FTM), both of which are commercially available (e.g., Difco). SCDB is conducive to growth of aerobic bacteria and fungi incubated at 20-25° C., while FTM is conducive to growth of aerobic and anaerobic bacteria incubated at 30-35° C. Cultures in both media are incubated for 14 days. Negative growth by the end of the incubation period confirms meeting the sterility requirements according to USP Standard 26: 2003 <71>.

As is illustrated in the Examples section which follows, stinging capsule preparations which have been treated with Gamma radiation (1 or 2 MRad), ultraviolet radiation (312 nm for 20 min), microwave radiation (8 sec exposure in a standard microwave), ethanol (70% for 10 min), hydrogen peroxide (1% for 5 min) or steam under pressure (121° C. for 20 min) meet the sterility criteria set forth by USP guidelines.

Stinging capsules preparations intended for topical application which may not require drastic sterilization treatment such as described above can be subjected to conditions which would eliminate most if not all microbial contaminants (as determined by a bioburden test, see below). Such conditions include, but are not limited to, freeze-drying, selective heat, selective irradiation or selective exposure to an antimicrobial agent. Stinging capsule preparation treated as such can be tested to meet the acceptable bioburden value for cosmetic or therapeutic compositions, or medical devise intended for topical application, in accordance with international regulations and guidelines. Most preferably, the stinging capsule preparation is treated under conditions which result in a bioburden value below 1 cfu/ml determined by using the standard method of ISO 11737 part 1 BFEN 2274-3.

As is mentioned hereinabove, sterile stinging capsule preparations are highly suitable for delivery of therapeutic, diagnostic or cosmetic agents into a host tissue.

Delivery of a therapeutic, diagnostic or cosmetic agent into a tissue can be effected by applying the sterile stinging capsule preparation, which includes at least one stinging capsule and the therapeutic, diagnostic or cosmetic agent, to an outer surface of the tissue (e.g., skin) which is preferably surface sterilized with a suitable solution (e.g., alcohol). Following application, the stinging capsules are triggered (as is further described hereinbelow) and the therapeutic, diagnostic or cosmetic agent is thereby delivered by the tubule into the tissue.

Alternatively, the therapeutic, diagnostic or cosmetic agent can be applied onto the outer surface of the tissue, followed by application of a sterile stinging capsule preparation to the same region. Upon triggering, the agent is pumped into the stinging capsules (as is further described herein) and the therapeutic, diagnostic or cosmetic agent is delivered via the tubule into the tissue.

Preferably, the therapeutic, diagnostic or cosmetic agent of the present invention is sterilized and is in compliance with international sterility testing standards.

The composition-of-matter of the present invention may include a mixture of at least one stinging capsule and a therapeutic, diagnostic or cosmetic agent being devoid of viable microorganisms. The composition may be fabricated by first combining the stinging capsule(s) with the agent followed by sterilizing the mixed product. Alternatively, the composition may be fabricated by first sterilizing the stinging capsule(s) and the agent separately, followed by aseptically combining the two components together. However, since suitable therapeutic, diagnostic or cosmetic agents may not always withstand drastic sterilization treatments deemed necessary to reliably sterilize stinging capsules, their sterilization is preferably effected apart from the stinging capsule preparation using compatible procedures such as, for example, membrane filtration.

According to preferred embodiments of the present invention, the therapeutic agent can be any biological active factor such as, for example, a drug, a nucleic acid construct, a vaccine, a hormone, an enzyme, small molecules such as for example iodine or an antibody. Examples include, but are not limited to, antibiotic agents, free radical generating agents, anti fungal agents, anti-viral agents, non-nucleoside reverse transcriptase inhibitors, protease inhibitors, non-steroidal anti inflammatory drugs, immunosuppressants, anti-histamine agents, retinoid agents, tar agents, anti-puritic agents, hormones, psoralen, and scabicide agents. Nucleic acid constructs deliverable by the present invention can encode polypeptides (such as enzymes ligands or peptide drugs), antisense RNA, or ribozymes.

The therapeutic agent can also be a prodrug, which is activatable prior to, during, or following discharge of the stinging capsule. As used herein in the specification and in the claims section which follows, the term “prodrug” refers to an agent which is inactive but which is convertible into an active form via enzymatic, chemical or physical activators.

A prodrug (for example an enzyme) can be activated just prior to stinging capsule discharge by providing an activator compound (for example an ion), which can be diffused or pumped (during discharge) into the capsule. Alternatively, specific enzymes, molecules or pH conditions present in the target tissues, can activate the prodrug.

The cosmetic agent of the present invention can be, for example, an anti-wrinkling agent, an anti-acne agent, a vitamin, a skin peel agent, a hair follicle stimulating agent or a hair follicle suppressing agent. Using the stinging capsules of the present invention a more effective delivery of such cosmetic agents can be effected. Examples of cosmetic agents include, but are not limited to, retinoic acid and its derivatives, salicylic acid and derivatives thereof, sulfur-containing D and L amino acids and their derivatives and salts, particularly the N-acetyl derivatives, alpha-hydroxy acids, e.g., glycolic acid, and lactic acid, phytic acid, lipoic acid and many other agents which are known in the art, such as, for example the hair follicle stimulating or suppressing agents described hereinbelow.

In addition, sterile stinging capsules capable of injecting a cosmetic dye can be utilized as a sterile, needle free and pain free method of producing permanent or transient tattoos. For such purposes, a predetermined pattern of stinging capsules can be attached to a support such as a plaster, foil or the like as described hereinabove. The stinging capsules can be preloaded with a cosmetic dye or immersed therein prior to, or during triggering activation (e.g., the cosmetic dye can be applied to the skin). Upon stinging capsules discharge (via, for example, skin contact), the dye would penetrate into the skin to form a predetermined dye pattern (tattoo).

According to one preferred embodiment of the present invention, the therapeutic, diagnostic or cosmetic agent is disposed within the liquid stored in the stinging capsule. In such a case, the capsule is loaded with the therapeutic, diagnostic or cosmetic agent via any one of several methods generally known in the art such as, but not limited to, diffusion, electroporation, liposome fusion, microinjection and the like.

Alternatively and according to another preferred embodiment of the present invention, the therapeutic, diagnostic or cosmetic agent is disposed in a liquid surrounding the stinging capsule. In such a case, the capsule's natural mechanism of osmotically collecting liquid from the environment following triggering (further detailed in the background section hereinabove) pumps the therapeutic, diagnostic or cosmetic agent into the stinging capsule just prior to or during the discharge.

In any case, since a stinging capsule is highly permeable to water and molecules, therapeutic, diagnostic or cosmetic agent loading prior to or during discharge can be easily achieved.

Prior art studies which concentrated on deciphering the permeability and functionality of stinging capsules have shown that alkali ions, monovalent ions, divalent ions, or small organic cations such as Tris+ or choline+, penetrate cnidocysts and accumulate inside without affecting the properties of the stinging capsule. Studies performed by Lubbock & Amos in order to understand the effect of calcium on capsule discharge (1981) have shown that in the predischarged state the cnida wall is permeable to water and to charged molecules of relatively low molecular weight like bromophenol blue (MW 670) and fluoresceinate (MW 376). Hidaka, who investigated of the mechanism of capsule discharge (1992, 1993) demonstrated that cnidocysts stained with toluidine blue (MW 306) released the blue stain through the tubule when discharged leaving the capsule completely clear. Heeger et al., (1992) investigated the ability of different commercially available lotions to protect human skin against stinging capsule penetration.

Thus, short polypeptides, hormones, or any low molecule weight agents can be loaded into stinging capsules through simple diffusion. These active compounds can be stored in the capsule and injected into the target tissue upon discharge.

As mentioned hereinabove, during the discharge process, the immediate liquid surrounding the stinging capsule is pumped into the capsule and than injected via the tubule. Since the surrounding liquid is pumped into the capsules under extremely high pressures over a short period of time it is highly plausible that high molecular weight molecules, such as polypeptides polynucleotides and other complex molecules can penetrate the capsule and be delivered via the tubule upon discharge.

In any case, the sterile stinging capsule preparations described above can be directly utilized to deliver the therapeutic, diagnostic or cosmetic agent into mammalian and other tissue by applying the stinging capsules isolated therefrom, which include the agent, or by co-applying the agent and stinging capsule onto a skin region of an individual (e.g. a human or livestock and other) and triggering discharge either automatically (via contact with the tissue) or manually via an activation mechanism which is described in detail hereinbelow.

Triggering the activation of the stinging capsules thus leads to the subsequent topical, transdermal/intradermal, transmucosal, transmembranal or transcuticular delivery of the therapeutic, diagnostic or cosmetic agent under aseptic conditions.

To stabilize the therapeutic, diagnostic or cosmetic agent and/or the stinging capsules and to possibly enhance triggering efficiency, the composition-of-matter of the present invention is preferably included in a pharmaceutical composition formulated for such purposes.

Thus, according to another aspect of the present invention there is provided a pharmaceutical composition, which includes, as an active ingredient, a therapeutic, diagnostic or cosmetic agent, at least one stinging capsule and a pharmaceutically acceptable carrier and which is devoid of viable microorganisms.

Hereinafter, the phrase “pharmaceutically acceptable carrier” refers to a carrier, which does not cause significant irritation to the individual treated and does not abrogate the biological activity and properties of the active ingredient.

Preferably, the carrier of the present invention is sterile. More preferably, the carrier is tested for sterility in compliance with USP and similar international sterility testing guidelines.

The sterile pharmaceutical composition of the present invention can be fabricated by either sterilizing the composition as a whole, or by separately sterilizing its components separately followed by mixing the sterilized components under aseptic conditions.

Preferably, the pharmaceutically acceptable carrier does not affect the ability of the stinging capsules to discharge following triggering although in some instances, a pharmaceutically acceptable carrier which inhibits triggering mediated by tissue contact can also be utilized by the present invention.

The pharmaceutical composition of the present invention includes a pharmaceutically acceptable carrier, which is formulated for topical, transmucosal or transnasal applications.

For topical application, the active ingredient and stinging capsule(s) may be suspended in hydrophilic or hydrophobic-based carrier such as a gel suitable for topical applications.

For topical, transmucosal or transnasal administration, the active ingredient and stinging capsules can be conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount.

In order to prevent accidental microbial contamination during prolong use or storage (for example, due to mishandling the product by the end user), the pharmaceutical composition of the present invention may further include one or more preservative compounds capable of preventing microbial growth. Suitable preservatives include, but not limited to, effective concentrations of ethanol, propylene glycol, benzyl alcohol, phenoxyethanol, diazolidinyl urea, parabens, sulfites, benzalkonium chloride chlorocresol, phenol, phenylethanol, chlorohexidine, thiomersal, benzoic acid and sorbic acid. Additional suitable preservatives are described in http://pharmacos.eudra.org/F3/inci/incif41.htm.

In any case, application of the composition to, for example, a skin region leads to subsequent contact between the stinging capsule(s) and the skin the individual which contact triggers discharge of the stinging capsule and delivery of the active ingredient into for example, an epidermis or dermis tissue region of the individual.

Although, for some applications, contact mediated discharge suffices, such activation can be inefficient since it enables discharge of only the portion of stinging capsules, which come into physical contact with the tissue following application.

To enable a more efficient and consistent discharge and thus delivery of the active agent, the pharmaceutical composition of the present invention can include a pharmaceutically acceptable carrier which inhibits discharge of the stinging capsule(s) upon tissue contact (e.g., skin contact). In such cases, the pharmaceutical composition also includes a chemical activator, such as, for example NaSCN or EGTA, which can be applied prior to, or following, application of the pharmaceutical composition and which triggers discharge of the stinging capsules. Chemical or electrical activation of discharge is advantageous since it allows for simultaneous discharge of most if not all of the stinging capsules of the pharmaceutical composition.

Chemical triggering can be mediated by substances such as free and conjugated N-acetylated sugars or low molecular weight amino compounds which are known to be detected by at least two classes of stinging cell chemoreceptors. As described in U.S. Pat. No. 6,613,344, Sodium thiocyanate (NaSCN) is capable of triggering discharge of cnidocysts. In addition, Lubbock and Amos (1981) have shown that isolated cnida (cnidocysts) can discharge normally when placed in buffered EGTA or 10 mM citrate solution; Weber (1989) demonstrated the effect of dithioerthritol or proteases on discharging isolated cnida and Hidaka (1993) discussed various agents which can trigger cnida discharge.

Electrical triggering can be achieved via an electrical pulse of 30 microseconds of approximately 20-30 Volts as is further described in the literature (Holstein and Tardent 1984; Tardent and Holstein 1982).

As mentioned hereinabove, the present invention can be utilized to deliver a variety of therapeutic agents. Such therapeutic agents combined with the effective delivery obtainable via capsules can be utilized to treat a variety of disorders.

An example of a very common skin infection is acne, which involve infestation of the sebaceous gland with P. acnes, as well Staphylococus aurus and pseudomonas. The disorder can be treated by anti-bacterial agents such as phenols, including cresols and resorcinols and antibiotics such as chloramphenicol, tetracyclines, synthetic and semi-synthetic penicillins, beta-lactames, quinolones, fluoroquinolnes, macrolide antibiotics, peptide antibiotics, cyclosporines, erytromycin and clindamycin.

Psoriasis, which is a common skin disorder can be treated by using the present invention for accurate and efficient intraepidermal delivery of steroidal anti-inflammatory agents or other known drugs with limited skin permeability.

Fungal infections can also be treated via the pharmaceutical composition of the present invention. Superficial fungal infection of the skin is one of the commonest skin diseases seen in general practice. Dermatophytosis is probably the most common superficial fungal infection of the skin. Candidiasis is an infection caused by the yeast like fungus candida albicans or occasionally other species of candida. Antifungal drugs, which are active against dermatophytes and candida such as azoles, diazoles, triazoles, miconazole, fluconazole, ketoconazole, clotrimazole, itraconazole griseofulvin, ciclopirox, amorolfine, terbinafine, Amphotericin B, potassium iodide, flucytosine (5FC) and any combination thereof at a therapeutically effective concentration can be delivered intraepidermally via the method of the present invention.

The present invention can be also used for delivering pigments, such as photosensitizers utilizable in photo dynamic therapy (PDT), into cells of skin cancer or other skin disorders. Photosensitizers are chemical compound which produce a biological effect upon photoactivation, or a biological precursor of a compound that produces a biological effect upon photoactivation. Examples of photosensitizers which can be delivered by the stinging capsules of the present invention include, but are not limited to, hematoporphyrins (Batlle 1993 J. Photochem. Photobiol. Biol. 20:5-22 and Kessel 1988 Cancer Let. 39:193-198), uroporphyrins and phthalocyanines (Kreimer-Bimbaum, 1989 Seminars in Hematology 26:157-173), purpurins (Morgan et al. 1990 Photochem. Photobiol. 51:589-592 and Kessel, 1989 Photochem. Photobiol. 50:169-174), acridine dyes and bacteriochlorophylls (Beems et al. 1987 Photochem. Photobiol. 46:639-643 and Kessel et al. 1989 Photochem. Photobiol. 49:157-160), and bacteriochlorins (Gurinovich et al. 1992 J. Photochem. Photobiol. Biol. 13:51-57).

By enabling accurate and efficient delivery of photosensitizers, the present invention substantially improves the efficiency of PDT.

Eye infections such as conjuctivitis, caused by bacteria such as staphylococcus aureus, streptococcus pneumoniae, and haemophilus influenzae can be treated with antibiotic ointments, e.g., bacitracin which is delivered via the method of the present invention.

Chronic rheumatic or arthritic conditions are usually treated by NSAIDs. Such as salicylic acid, or aspirin, and ibuprofen are well-known examples of NSAID drugs. Patients taking NSAIDs drugs orally face an increased risk for peptic ulcers and gastrointestinal blood loss resulting in anaemia. Such adverse reactions especially plague patients taking NSAIDs drugs over prolonged periods. Transdermal administration of NSAIDs via the delivery device or method of the present invention will prevent the gastrointestinal complications. Transdermal drug delivery according to the present invention provides other benefits such as less frequent dosing; better controlled drug release, and a greater ability to target delivery to specific tissue sites.

Anaesthetics can be used for alleviating pain for example during suturing, or in infections, which are accompanied with pain sensation. Examples of topical anaesthetic drugs include without limitation benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof all of which are deliverable via the delivery device or method of the present invention.

The sterile stinging capsule preparation of the present invention can be used to treat hair loss, excessive hair growth, or discoloration of the hair.

For example, a hair follicle stimulating agent such as hinokitiol, or pantothenic acid can be delivered by the stinging capsules of the present invention directly into the follicle in order to stimulate hair growth.

Allternatively, the sterile stinging capsule preparation of the present invention can be utilized to deliver, directly into hair follicles, an hair follicle suppressing agent capable of suppressing hair growth. Examples of agents capable of suppressing hair growth include, but are not limited to, non-steroidal suppressors of angiogenesis and inhibitors of 5-alpha reductase, ornithine decarboxylase, S-adenosylmethionine decarboxylase, gamma-glutamyl transpeptidase, and transglutaminase.

The present invention can also be utilized to pigment hair color by delivering, for example, melanin or tyrosinase, into the hair follicle.

In addition to the above, the teachings of the present invention can also be utilized to deliver drugs into blood circulation via either transdermal delivery, which leads to diffusion into small capillaries or by applying the delivery device of the present invention to internal body tissues.

In such cases, the present invention can be utilized to deliver drugs such as hormones (e.g., insulin), antibiotics, cardiac drugs and the like.

The sterile stinging capsule preparation of the present invention can also be utilized for vaccination. Vaccine antigens can be delivered to specialized immune cells underlying the skin or into blood circulation (as described above).

Absorption into the blood stream following transdermal delivery will most likely result in transport of the antigen to the phagocytic cells of the liver, spleen, and bone marrow. Since such cells serve as antigen presenting cells, a strong immunogenic response will be elicited leading to effective immunization.

Thus, the present invention overcomes the limitations of prior art devices and methods while providing a safe, efficient and contamination risk free method for delivering agents across stratum corneum, epidermal mucousal or membranal barriers.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non-limiting fashion.

### Example 1

### Sterilization of Stinging Capsules

Materials and Methods:

Isolation of Capsules:

Fresh tissue of the sea anemone Aiptasia diaphana were homogenized in sodium citrate as described in Salleo et al. (Physiol Zool 61: 272-279, 1988). The homogenate (300 μl) was added to percol (300 μl) in a microfuge tube. The tube was shaken over ice for 30 min and then centrifuged for 10 minutes, at 1000 rpm. The pellet was washed 3 times with H₂O and re-suspended in 50 μl H2O as a liquid preparation. Alternatively, the pellet was frozen in liquid nitrogen or −80° C. and than freeze dried as a dry preparation.

Sterilization Treatments:

Gamma radiation: isolated sea anemone (Aiptasia diaphana) and jellyfish (Rhopilema numadica) capsules in dry preparation were exposed to gamma radiation of 1 and 2 MRad.

Ultraviolet radiation: isolated sea anemone Aiptasia diaphana, jellyfish (Rhopilema nomadica) and hydra (Hydra vulgaris) in suspension were exposed to UV radiation at 312 nm for 20 minutes.

Microwave radiation: isolated sea anemone capsules in dry preparation were exposed to standard microwave radiation for 8 seconds.

Moist heat: isolated hydra capsules were autoclaved at 121° C. for 20 minutes.

Ethanol: isolated sea anemone and jellyfish capsules were exposed to 70% ethanol for 10 minutes then centrifuged at 1000 g for 10 minutes. Following centrifugation, the supernatant was discarded and the pellet, comprising the treated stinging capsules, was air dried in a sterile laminar flow hood.

Hydrogen peroxide: isolated sea anemone and jellyfish capsules were exposed to 1% hydrogen peroxide for 5 minutes, then centrifuged at 1000 g for 10 minutes and washed twice in sterile solution.

Sterility Testing: Six samples representing about 10% of the product lot were tested for sterility. Each sample (approximately 10⁶ capsules) was inoculated into a flask containing Soybean Casein Digest Broth (SCDB) and into a flask containing of Fluid Thioglycollate Medium (FTM). Cultures of Bacillus subtilis, Candida albicans and Aspergillus niger were inoculated into SCBD as positive controls. Cultures of Clostridium sporogenes, Pseudomonas auriginosa and Staphylococcus aureus were inoculated into FTM as positive controls. All inoculated SCDB flasks were incubated in a shaker incubator at 25-30° C. and for 14 days. All inoculated FTM flasks were incubated at 30-35° C. for 14 days. Positive growth was determined by an appearance of turbidity in the culture medium by the end of incubation.

Limulus amebocyte lyase (LAL) test: The LAL test, intended for bacterial endotoxin detection in medical devises, was performed as described by Levin and Bang Bull Johns Hopkins Hosp. 115:265-74, 1964)

Testing of the stinging (discharging) capacity of sterilized capsules: capsule suspension samples (2 μl, approximately 1000 capsules) were applied to a microscope slide followed by adding sodium thiocyanate (NaSCN, 2 μl). An immediate tubules discharge from capsules, observed under a light microscope (Leitz Laborlux S), was indicative of a positive activity of capsules.

Results:

Sterility of capsules preparation following freeze drying: Five samples of freeze dried capsules (10⁶ each) were pooled and cultured on SCDB and FTM. Following 14 days of incubation at 25° C. less than 1 cfu/ml developed on the growth media, indicating a bioburden-acceptable preparation.

Preliminary evaluation of sterility of capsules: capsules were inoculated in LB medium (Difco) and incubated at 37° C. for 48° C. By the end of incubation no turbidity was observed in the culture medium indicating lack of bacterial growth in capsules preparation.

LAL test: Four different lots of stinging capsule preparations were evaluated. Three samples were obtained from each lot and tested using the clot gel technique. All tests resulted in less than 0.5 EU/sample indicating that no endotoxin was present in the tested preparations.

Sterility of capsules preparation following exposure to gamma radiation, UV radiation, microwave radiation, moist heat, ethanol or hydrogen peroxide: No turbidity was observed in test media of all tested samples by the end of 48 hr incubation period in LB, indicating sterility of all treated preparations.

Stinging (discharging) capacity of sterilized capsules: capsule preparations which were sterilized by exposure to gamma-irradiation, UV irradiation, microwave, moist heat, ethanol or hydrogen peroxide retained their full tubule discharging capacity, as compared with similar non-sterilized capsules.

The results show that a variety of sterilization treatments can be effectively applied to stinging capsules preparations without affecting their discharging (stinging) capacity. The sterilized capsules can be safely utilized for delivering therapeutic, diagnostic or cosmetic agents into subcuticular tissues without any risk of infection.

### Example 2

### Compatibility of Capsules in Formulations Containing Anti-Microbial Agents

Materials and Methods:

In order to prevent microbial contamination of isolated stinging capsules preparations, the capacity of capsules to endure exposure to several known compounds and preservatives has been evaluated. Accordingly, stinging capsules isolated from Aiptasia diaphana as described in Example 1, were exposed for at least 1 month up to 1 year to the following compositions:
(a) Gel formulation comprising 97% ethanol and 3% hydroxylpropyl cellulose.
(b) Liquid formulation comprising 75% ethanol and 25% Cetiol.
(c) Gel formulation comprising 95% propylene glycol and 0.5% Carbopol ETD2020.
(d) Gel formulation comprising 97% propylene glycol and 3% PVP.
(e) Gel formulation comprising 97% propylene glycol and 1.5% benzyl alcohol

Results:

The discharging capacity of treated capsules was determined using the procedure as described in Example 1. None of the tested compositions affected the discharging capacity of capsules under the experimental conditions. Thus, the results show that a variety of preservatives capable of preventing microbial contamination can be utilized in a pharmaceutical composition including stinging capsules.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications disclosed therein and/or mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

1. Anderson, C. L.; Canning, E. U., and Okamura, B. (1998). “A triploblast origin for Myxozoa?” Nature, 392(6674), 346-7.
2. Brennecke, T., Gellner, K., and Bosch, T. C. (1998). “The lack of a stress response in Hydra oligactis is due to reduced hsp70 mRNA stability.” Eur J Biochem, 255(3), 703-9.
3. Godkenecht, A., and Tardent, P. (1988). “Discharge and mode of action of the tentacular nematocysts of Anemonia sulcata (Antozoa: Cnidaria).” Marine Biology, 100, 83-92.
4. Heeger, T., Moller, H., and Mroweitz, U. (1992). “Protection of human skin against jellyfish (Cyanea capillata) stings.” Marine Biology, 113, 669-678.
5. Hidaka, M. (1992). “Effects of Ca+ on the volume of nematocysts isolated from acontia of the sea anemone Calliactis tricolor.” Comp Biochem Physiol, 101A(4), 737-741.
6. Hidaka, M. (1993). “Mechanism of nematocystn discharge and its cellular control.” Advances in Comparative and Environmemtal Physiology, 15, 45-76.
7. Holstein, T., and Tardent, P. (1984). “An ultrahigh-speed analysis of exocytosis: nematocyst discharge.” Science, 223(4638), 830-3.
8. Lotan, A., Fishman, L., Loya, Y., and Zlotkin, E. (1995). “Delivery of a nematocyst toxin Nature, 375(6531), 456.
9. Lotan, A., Fishman, L., and Zlotkin, E. (1996). “Toxin compartmentation and delivery in the Cnidaria: the nematocyst's tubule as a multiheaded poisonous arrow.” J Exp Zool, 275(6), 444-51.
10. Lubbock, R. (1979). “Chemical recognition and nematocyte exitation in sea anemone.” J. exp. Biol., 83, 283-292.
11. Lubbock, R., and Amos, W. B. (1981). “Removal of bound calcium from nematocyst contents causes discharge.” Nature, 290(5806), 500-1.
12. Mariscal, R. N. (1974). Coelenterate biology: reviews and new perspectives, Academic Press, New York.
13. Siddall, M. E., Martin, D. S., Bridge, D., Desser, S. S., and Cone, D. K. (1995). “The demise of a phylum of protists: phylogeny of Myxozoa and other parasitic cnidaria.” J Parasitol, 81(6), 961-7.
14. Smothers, J. F., von Dohlen, C. D., Smith, L. H., Jr., and Spall, R. D. (1994). “Molecular evidence that the myxozoan protists are metazoans.” Science, 265(5179), 1719-21.
15. Tardent, P. (1995). “The cnidarian cnidocyte, a high-tech cellular weaponry.” BioEssays, 17(4), 351-362.
16. Tardent, P., and Holstein, T. (1982). “Morphology and morphodynamics of the stenotele nematocyst of Hydra attenuata Pall. (Hydrozoa, Cnidaria).” Cell Tissue Res, 224(2), 269-90.
17. Thorington, G. U., and Hessinger, D. A. (1988). “Control of cnida discharge: I. evidence for two classes of chemoreceptor.” Biol. Bull., 174, 163-171.
18. Watson, G. M., and Hessinger, D. (1989). “Cnidocyte mechanoreceptors are tuned to the movements of swimming prey by chemoreceptors.” Science, 243, 1585-1591.
19. Watson, G. M., and Hessinger, D. A. (1992). “Receptors for N-acetylated sugars may stimulate adenylate cyclase to sensitize and tune mechanoreceptors involved in triggering nematocyst discharge.” Exp Cell Res, 198(1), 8-16.
20. Weber, J. (1989). “Nematocysts (stinging capsules of Cnidaria) as Donnan-potential-dominated osmotic systems.” Eur J Biochem, 184(2), 465-76.
21. Westfall, J. A., Bradbury, P. C., and Townsend, J. W. (1983). “Ultrastructure of the dinoflagellate Polykrikos. I. Development of the nematocyst-taeniocyst complex and morphology of the site for extrusion.” J Cell Sci, 63, 245-61.

What is claimed is:

1. A composition-of-matter comprising a sterile stinging capsule preparation including at least one stinging capsule.

2. The composition-of-matter of claim 1, wherein said sterile stinging capsule preparation is devoid of a microorganism capable of growing in a microbiological culture medium.

3. The composition-of-matter of claim 2, wherein said microbiological culture medium is soybean casein digest broth (SCDB) or fluid thioglycollate medium (FTM).

4. The composition-of-matter of claim 1, further comprising a therapeutic, diagnostic or cosmetic agent.

5. The composition-of-matter of claim 4, wherein said therapeutic, diagnostic or cosmetic agent is disposed in a liquid surrounding, or stored within, said at least one stinging capsule.

6. The composition-of-matter of claim 4, wherein said cosmetic agent is selected from the group consisting of a cosmetic dye, an anti wrinkling agent, an anti-acne agent, a vitamin, a skin peel agent, a hair follicle stimulating agent and a hair follicle suppressing agent.

7. The composition-of-matter of claim 4, wherein said therapeutic agent is selected from the group consisting of a drug, a nucleic acid construct, a vaccine, a hormone, an enzyme and an antibody.

8. The composition-of-matter of claim 4, wherein said therapeutic agent is a prodrug activatable prior to, during or following discharge of said at least one sterile stinging capsule.

9. The composition-of-matter of claim 4, wherein said at least one stinging capsule is capable of delivering said therapeutic, diagnostic or cosmetic agent into a tissue.

10. The composition-of-matter of claim 1, wherein an endogenous toxin naturally stored within said at least one stinging capsule is substantially non-toxic to mammals.

11. The composition-of-matter of claim 10, wherein said endogenous toxin is non-functional.

12. The composition-of-matter of claim 1, wherein said at least one stinging capsule is derived from an organism of a class selected from the group consisting of Anthozoa, Hydrozoa and Scyphozoa.

13. The composition-of-matter claim of 1, wherein said at least one stinging capsule is derived from an organism of a phylum selected from the group consisting of Cnidaria, Dinoflagellata and Myxozoa.

14. The composition-of-matter of claim 1 further comprising an effective amount of at least one preservative capable of preventing microbial growth within the sterile stinging capsule preparation.

15. The composition-of-matter of claim 14, wherein said at least one preservative is selected from the group consisting of ethanol, propylene glycol, benzyl alcohol, phenoxyethanol, diazolidinyl urea, parabens, sulfites, benzalkonium chloride, chlorocresol, phenol, phenylethanol, chlorohexidine, thiomersal, benzoic acid and sorbic acid.

16. A pharmaceutical composition comprising, as an active ingredient, a stinging capsule preparation including at least one stinging capsule and a pharmaceutically acceptable carrier, the pharmaceutical composition being devoid of viable microorganisms.

17. The pharmaceutical composition of claim 16, wherein said stinging capsule preparation further comprising a therapeutic, diagnostic or cosmetic agent.

18. The pharmaceutical composition of claim 17, wherein said therapeutic, diagnostic or cosmetic agent is disposed in a liquid surrounding, or stored within, said at least one stinging capsule.

19. The pharmaceutical composition of claim 17, wherein said cosmetic agent is selected from the group consisting of a cosmetic dye, an anti-wrinkling agent, an anti-acne agent, a vitamin, a skin peel agent, a hair follicle stimulating agent and a hair follicle suppressing agent.

20. The pharmaceutical composition of claim 17, wherein said therapeutic agent is selected from the group consisting of a drug, a nucleic acid construct, a vaccine, a hormone, an enzyme and an antibody.

21. The pharmaceutical composition of claim 17, wherein said therapeutic agent is a prodrug activatable prior to, during or following discharge of said at least one stinging capsule.

22. The pharmaceutical composition of claim 17, wherein said at least one stinging capsule is capable of delivering said therapeutic, diagnostic or cosmetic agent into a tissue.

23. The pharmaceutical composition of claim 16, wherein an endogenous toxin stored within said at least one stinging capsule is substantially non-toxic to mammals.

24. The pharmaceutical composition of claim 23, wherein said endogenous toxin is non-functional.

25. The pharmaceutical composition of claim 16, wherein said at least one stinging capsule is derived from an organism of a class selected from the group consisting of Anthozoa, Hydrozoa and Scyphozoa.

26. The pharmaceutical composition of claim 16, wherein said at least one stinging capsule is derived from an organism of a phylum selected from the group consisting of Cnidaria, Dinoflagellata and Myxozoa.

27. The pharmaceutical composition of claim 16, wherein said pharmaceutically acceptable carrier is selected from the group consisting of an aqueous solution, spray, a gel, an oil, an ointment and semi solid formulation.

28. The pharmaceutical composition of claim 16 further comprising an effective amount of a preservative agent capable of preventing microbial growth within the sterile pharmaceutical composition.

29. The pharmaceutical composition of claim 18, wherein said preservative agent is selected from the group consisting of ethanol, propylene glycol, benzyl alcohol, phenoxyethanol, diazolidinyl urea, parabens, sulfites, benzalkonium chloride, chlorocresol, phenol, phenylethanol, chlorohexidine, thiomersal, benzoic acid and sorbic acid.

30. A method of preparing a sterile stinging capsule preparation, comprising:
(a) providing a preparation including at least one stinging capsule; and
(b) subjecting said preparation to sterilizing conditions to thereby obtain the sterile stinging capsule preparation.

31. The method of claim 30, wherein said organism is of a class selected from the group consisting of Anthozoa, Hydrozoa and Scyphozoa.

32. The method of claim 31, wherein said organism is of a phylum selected from the group consisting of Cnidaria, Dinoflagellata and Myxozoa.

33. The method of claim 30, wherein step (b) is effected by a physical sterilization method.

34. The method of claim 33, wherein said physical sterilization method is selected from the group consisting of an exposure to moist heat, an exposure to dry heat, an exposure to gamma radiation, an exposure to ultraviolet radiation and an exposure to microwave radiation.

35. The method of claim 30, wherein said inactivating is effected by a chemical sterilization method.

36. The method of claim 35, wherein said chemical sterilization method is selected from the group consisting of an exposure to ethylene oxide, an exposure to ethanol and an exposure to hydrogen peroxide.

37. A method of delivering a therapeutic, diagnostic or cosmetic agent into a tissue, comprising:
(a) applying to the tissue a pharmaceutical composition including:
(i) a sterile stinging capsule preparation including at least one stinging capsule; and
(ii) the therapeutic, diagnostic or cosmetic agent; and
(b) triggering a discharge of said at least one stinging capsule to thereby deliver the therapeutic, diagnostic or cosmetic agent into the tissue.

38. The method of claim 37, wherein an endogenous toxin stored within said at least one stinging capsule is substantially non-toxic to mammals.

39. The method of claim 38, wherein said endogenous toxin is non-functional.

40. The method of claim 37, wherein said at least one stinging capsule is derived from an organism of a class selected from the group consisting of Anthozoa, Hydrozoa and Scyphozoa.

41. The method of claim 37, wherein said at least one stinging capsule is derived from an organism of a a phylum selected from the group consisting of Cnidaria, Dinoflagellata and Myxozoa.

42. The method of claim 37, wherein said therapeutic agent is selected from the group consisting of a drug, a nucleic acid construct, a vaccine, a hormone, an enzyme and an antibody.

43. The method of claim 37, wherein said therapeutic agent is a prodrug activatable prior to, during or following discharge of said at least one stinging capsule.

44. The method of claim 37, wherein said cosmetic agent is selected from the group consisting of a cosmetic dye, an anti-wrinkling agent, a vitamin, a skin peel agent, a hair follicle stimulating agent and a hair follicle suppressing agent.

45. The method of claim 37, wherein said triggering is effected by a change in pH, a chemical substance, a mechanical force or contact between said at least one sterile stinging capsule and said outer surface of the tissue.

46. A method of delivering a therapeutic, diagnostic or cosmetic agent into a tissue, comprising:
(a) applying onto an outer surface of the tissue a therapeutic, diagnostic or cosmetic agent;
(b) applying onto said outer surface of the tissue a sterile stinging capsule preparation including at least one stinging capsule; and
(c) triggering a discharge of said at least one stinging capsule to thereby deliver the therapeutic, diagnostic or cosmetic agent into the tissue.

47. The method of claim 46, wherein an endogenous toxin stored within said at least one stinging capsule is substantially non-toxic to mammals.

48. The method of claim 47, wherein said endogenous toxin is non-functional.

49. The method of claim 47, wherein said at least one stinging capsule is derived from an organism of a class selected from the group consisting of Anthozoa, Hydrozoa and Scyphozoa.

50. The method of claim 47, wherein said at least one stinging capsule is derived from an organism of a phylum selected from the group consisting of Cnidaria, Dinoflagellata and Myxozoa.

51. The method of claim 47, wherein said therapeutic agent is selected from the group consisting of a drug, a nucleic acid construct, a vaccine, a hormone an enzyme and an antibody.

52. The method of claim 47, wherein said therapeutic agent is a prodrug activatable prior to, during or following discharge of said at least one stinging capsule.

53. The method of claim 47, wherein said cosmetic agent is selected from the group consisting of a cosmetic dye, a vitamin, a skin peel agent, a hair follicle stimulating agent and a hair follicle suppressing agent.

54. The method of claim 47, wherein said triggering is effected by a change in pH, a chemical substance, a mechanical force or contact between said at least one stinging capsule and said outer surface of the tissue.

55. A stinging capsule preparation having a bioburden value lower than 1 cfu/ml determined by using the standard method of ISO 11737 part 1 BFEN 2274-3.

56. The stinging capsule preparation of claim 55, further comprising a therapeutic, diagnostic or cosmetic agent.

57. The stinging capsule preparation of claim 55, wherein said therapeutic, diagnostic or cosmetic agent is disposed in a liquid surrounding, or stored within, said at least one stinging capsule.

58. The stinging capsule preparation of claim 55, wherein said cosmetic agent is selected from the group consisting of a cosmetic dye, an anti wrinkling agent, an anti-acne agent, a vitamin, a skin peel agent, a hair follicle stimulating agent and a hair follicle suppressing agent.

59. The stinging capsule preparation of claim 55, wherein said therapeutic agent is selected from the group consisting of a drug, a nucleic acid construct, a vaccine, a hormone, an enzyme and an antibody.

60. The stinging capsule preparation of claim 55, wherein said therapeutic agent is a prodrug activatable prior to, during or following discharge of said at least one sterile stinging capsule.

61. The stinging capsule preparation of claim 55, wherein said at least one stinging capsule is capable of delivering said therapeutic, diagnostic or cosmetic agent into a tissue.

62. The stinging capsule preparation of claim 55, wherein an endogenous toxin naturally stored within said at least one stinging capsule is substantially non-toxic to mammals.

63. The stinging capsule preparation of claim 55, wherein said endogenous toxin is non-functional.

64. The stinging capsule preparation of claim 55, wherein said at least one stinging capsule is derived from an organism of a class selected from the group consisting of Anthozoa, Hydrozoa and Scyphozoa.

65. The stinging capsule preparation of claim 55, wherein said at least one stinging capsule is derived from an organism of a phylum selected from the group consisting of Cnidaria, Dinoflagellata and Myxozoa.

66. The stinging capsule preparation of claim 55 further comprising an effective amount of at least one preservative capable of preventing microbial growth within the sterile stinging capsule preparation.

67. The stinging capsule preparation of claim 55, wherein said at least one preservative is selected from the group consisting of ethanol, propylene glycol, benzyl alcohol, phenoxyethanol, diazolidinyl urea, parabens, sulfites, benzalkonium chloride, chlorocresol, phenol, phenylethanol, chlorohexidine, thiomersal, benzoic acid and sorbic acid.
